(19) United States
(12) Patent Application Publication
(10) Pub. No.: US 2025/0120645 A1
(43) Pub. Date: Apr. 17, 2025

(54) METHOD AND SYSTEM FOR ACQUIRING SPECTRAL DATA FROM HEALTHY BREAST TISSUE FOR USE IN DETECTING BREAST CANCER DEVELOPMENT, AND OTHER ORGAN TISSUE

(71) Applicant: DatChem, Brisbane City (AU)

(72) Inventors: Carolyn Mountford, Varsity Lakes (AU); Peter Malycha, St Georges (AU); John M. Irvine, Somerville, MA (US); Gorane Santamaria Hormaechea, Bowen Hills (AU); Darren Lukas, Surabaya (ID)

(73) Assignee: DatChem, Brisbane City (AU)

(21) Appl. No.: 18/916,171

(22) Filed: Oct. 15, 2024

Related U.S. Application Data
(60) Provisional application No. 63/589,966, filed on Oct. 12, 2023.

(51) Int. Cl. A61B 5/00; A61B 5/055; G01R 33/465

(52) U.S. Cl. CPC A61B 5/4312; A61B 5/004; A61B 5/055; A61B 5/7275; G01R 33/465

(57) ABSTRACT

A system and method enables the determination of the probably that a subject has or will develop breast cancer by obtaining spectral data of a selected biomarker from a first region of the subject's breast of presumably healthy breast tissue, the level of the biomarker having a correlation with the presence or probability of development of a breast tumor in a second region of the breast difference from the first region. The system and method may also be used for other organs, such as brain, ovary, kidney, and prostate.

### CROSS-REFERENCE TO RELATED APPLICATIONS

This application claims priority to U.S. Application Ser. No. 63/589,966 filed Oct. 13, 2023. Incorporated by reference herein.

### TECHNICAL FIELD

The present invention is directed to a method and system for acquiring spectral data from apparently healthy breast tissue or tissue of other organs using in vivo magnetic resonance spectroscopy (MRS) which can be used to detect early breast cancer or other organ cancer development (Switched On Tissue) different from the healthy tissue and not discernable by other imaging methods.

### BACKGROUND OF THE INVENTION

The application will discuss use on breast tissue as an example, but can apply to other organs, such as brain, ovary and kidney. There is an important need in the healthcare field to reliably and easily assess whether a woman has breast cancer in the form of a tumor. A common way is for a woman to obtain a mammography, which is typically done periodically especially in later years of age.

The categorical density of breast tissue can be obtained using a mammography. There are four breast composition categories for categorizing the levels of breast density according to the BI-RADS reporting system. The four levels are A: almost entirely fatty, B: scattered areas of fibroglandular density, C: heterogeneously dense which may obscure small masses, and D: extremely dense which lowers sensitivity of a mammography. Historically, women subjects have been distributed according to the following percentages in the four categories A: 10%, B: 40%, C: 40%, and D: 10%. Women subjects with higher density are typically at higher risk to develop breast cancer.

While breast cancer is typically detected through mammography, this modality still has false negatives and false positives. One source of false positives is due to dense and extremely dense breast tissue in categories C: and D:, which may obscure small masses and lowers the sensitivity of mammography.

One patent application, U.S. Publication No. US-2022-0202374-A1, incorporated by reference herein, relates to a method and system for assessing the risk of breast cancer by detecting the level of a tumor promotor methylmalonic acid (MMA), noting that higher levels indicate increased risk of developing breast cancer.

### SUMMARY OF THE INVENTION

According to the present invention, breast cancer tumors and the risk of developing same may be detected by obtaining spectral data of mammographically healthy breast tissue, and by correlating high levels of selected bio-markers from the spectral data with the presence of tumors in the breast in a region different from where the spectral data was obtained. Specifically, the present invention provides a mechanism to detect early development, not from examining the breast region where a tumor may exist and be directly detected, but from evaluating spectral data from an otherwise presumed or apparent healthy region of the breast, which spectral data is moving towards that of the presence of a tumor outside of the presumably healthy breast region. The presumed healthy tissue provides an indication that the other tissue in the breast is “switched on” and likely has the precursors of a tumor, from not even obtaining spectral data from the region where the tumor exists. This “switched on” tissue can be found in some who are considered high risk, and those considered not at risk prior to the changes being obvious using mammography, ultrasound or MRI.

The system and method can be used to monitor treatment of a subject. Very small foci or occult cancers can be treated with drugs. How a drug affects the tissue chemistry can be followed on an individual basis.

An in vivo examination of the chemistry of human breast tissue using MRS without the need for any contrast agent, can:
1. Determine breast density including fatty tissue from dense breast tissue including categories A:, B:, C:, and D:;
2. Distinguish the extent of deviation from normal healthy breast tissue in each of the categories in an individual;
3. Distinguish the “apparently healthy tissue” in a tumor bearing patient from normal healthy breast in each of the categories; the “apparently healthy tissue” is the full state of deviation from normal and constitutes an environment where a tumor can be developed or has developed but is not yet a mass lesion or is too small to see or detect using existing technology. There is a gradation recorded from fully healthy breast, to “switched on,” and then to a histologically and imaging recognizable cancer.

Spectral data used can be obtained using 1D and/or 2D MRS. The special data can be analyzed by:
a. Measuring the resonances or cross-peaks,
b. Data mining each digital point in a 1D spectrum, or
c. Data mining each digital point in both frequencies in a 2D spectrum

Using the above spectral data acquisition methods, classifiers can be used to automate the process. There are two approaches for the classifier:
1. A cascade classifier that first partitions into the fatty or dense breast then to subcategories of healthy, Switched On, or tumor.
2. A combination that does this in a single step classifier.

The information obtained can be used to monitor individual women over time to record dysregulation and thus risk, and manage the risk.

According to the invention, the risk for breast cancer can be determined by comparing the new patient breast tissue chemistry (of presumably healthy, i.e., tumor-free patients) measured by evaluation of the 2D COSY results and comparison with reference databases from the following cohorts:
A1. Breast density (fatty breast and dense breast), based on BI-RADS classification[1]: category A (fatty breast tissue), category B (scattered density), category C (heterogeneous density) and category D (extremely dense breast tissue);
A2: Presence and level of the tumor promotor methylmalonic acid (MMA) [2], capable of inducing the human transcription factor SOX4 expression and, consequently, elicit transcriptional reprogramming to give cancer cells aggressive properties[3];
A3: Chemical profile of the high risk tumor free (as determined by conventional imaging) cohort;
A4: Chemical profile of the apparently healthy tissue (determined by current imaging modalities), of a breast tumor bearing patient; and
A5: Classifiers developed from these data sets to automatically evaluate the new patient data by comparison to the cohorts listed.

### BRIEF DESCRIPTION OF THE DRAWINGS

FIG. 1 is a block diagram of a system which can be used to acquire and process spectral data of a subject including a magnetic resonance spectroscopy device for acquiring spectral data, and a comparator and analyzer for processing the data;

FIG. 2 is a flowchart showing TWIX (true raw data before any processing) and or DICOM (Digital Imaging and Communications in Medicine) data pre-processing of spectral data, which includes the steps of loading files on scanner, coil combination, frequency and phase correction, and averaging, followed by uploading data to the cloud;

FIG. 3 is a flowchart of a method for acquiring and processing 1D spectral data which supports reading and processing Siemens TWIX, DICOM, RDA (Siemens proprietary format of data that has undergone various levels of pre-processing on a scanner) files, general MRS pipeline, completely configurable for different use cases (e.g. brain, breast, ovary, kidney, and prostate), processes data, and performs quality assessment at every stage of pipeline;

FIG. 4 is a flowchart of the steps of a method for acquiring and processing 2D MR spectral data from a subject using Felix 2D Data measured by an individual which supports reading and processing Siemens TWIX and DICOM, general MRS pipeline, completely configurable for different use cases (e.g., brain, breast, ovary, kidney, prostate), processes data, and performs quality assessment at every stage of the pipeline, showing input of in cloud operation, residual H2O subtraction, apodization, zero filling, peak adjustment, and processing of 2D cosy data;

FIG. 5 is a flowchart showing classifier development from pipeline processed data, with cross-validation) including steps of inputting processed data, feature extraction, cross validation, including data imputation, scaling (normalization and standardization, oversampling (ROSE, tune dispersion), feature selection (sequential forward selection, selection from decision tree, cossvalidation to find N features to select, and metric balanced accuracy), classifier training (decision tree, LDA, 80% of data) and classifier test 120% of data);

FIG. 6 shows charts of results of fatty vs. dense breast separately in healthy control subjects, which show that 18 peaks have strong correlation, and BIRADS 1 & 2 (fatty breast) are similar, but BIRADS 3 & 4 (dense breast) are noticeably higher;

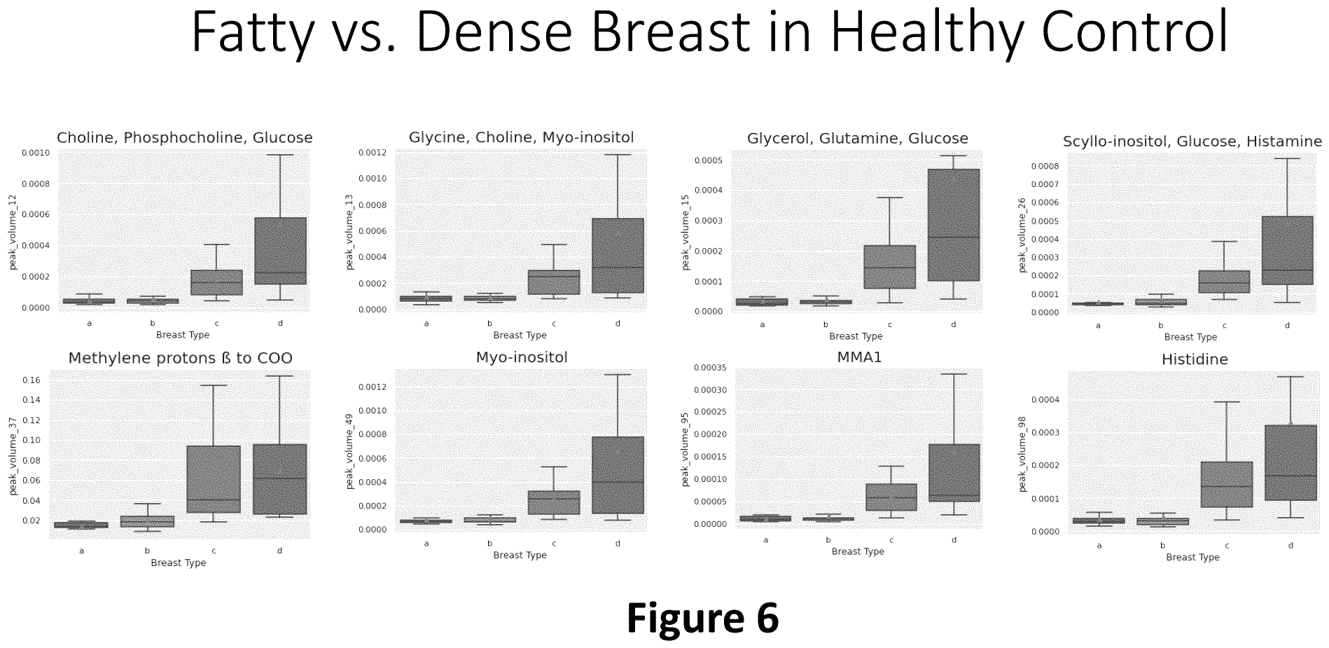

FIG. 7 is a chart comparing data for different classifiers and their accuracy, for dense vs. fatty breast

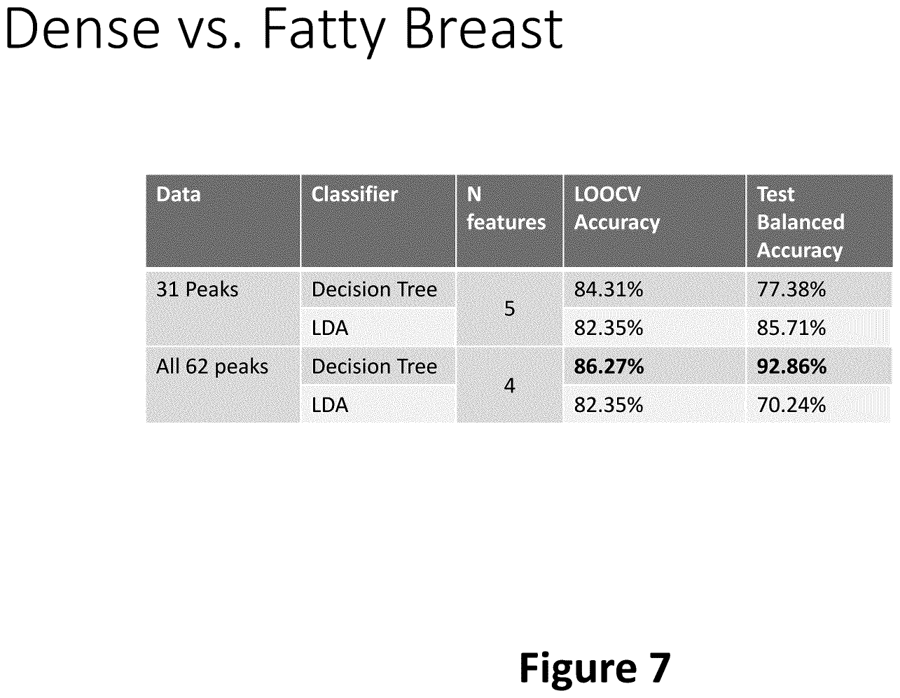

FIG. 8 is a chart showing results of fatty vs. dense breast tissue in high-risk patients for four peaks: taurine with strong correlation and 11 peaks with moderate correlation, with BIRADS type bands b and c having similar results and BIRADS type d having significantly higher peaks;

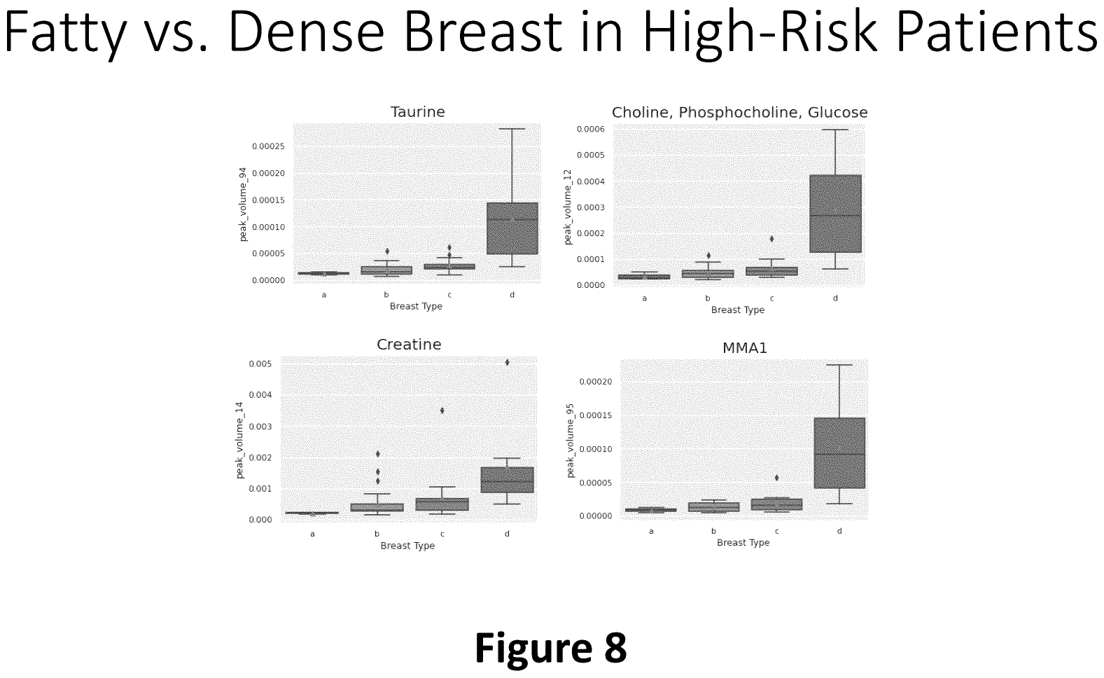

FIG. 9 is a graph of data showing data points of subjects with healthy breast fatty tissue, switched on in apparently healthy tissue, and tumor.

### DETAILED DESCRIPTION OF THE PREFERRED EMBODIMENTS

One or more preferred embodiments will be described, to the extent not already described above, but these embodiments are exemplary only and the invention is not limited to these embodiments.

As used herein, the subjects were human females, and have been referred to interchangeably as “subjects,” “females,” “women,” and “patients,” but could also be biologically males.

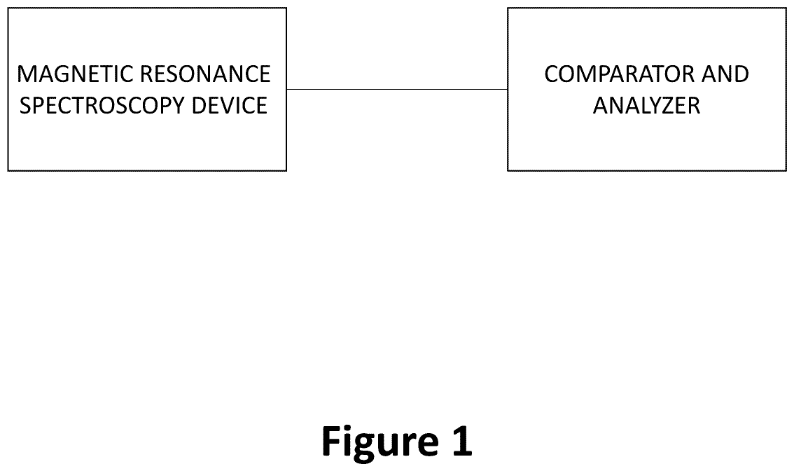

An apparatus or system for practicing the invention is shown in FIG. 1, which can be a 3T MRI machine which can be operated in a spectroscopy mode. The system includes memory, data analysis, comparators, as well as appropriate input/output interfaces. The classifiers may be in the cloud.

As described above, the spectral data can be obtained and recorded using 1D and/or 2D MRS, and the data can be analyzed by measuring the resonances or cross-peaks, by data mining each digital point in a 1D spectrum or data mining each digital point in both frequencies in a 2D spectrum. Classifiers can be developed to which new patient data can be acquired forming part of the reference database. The information can be used to monitor individual women over time to record dysregulation and thus risk.

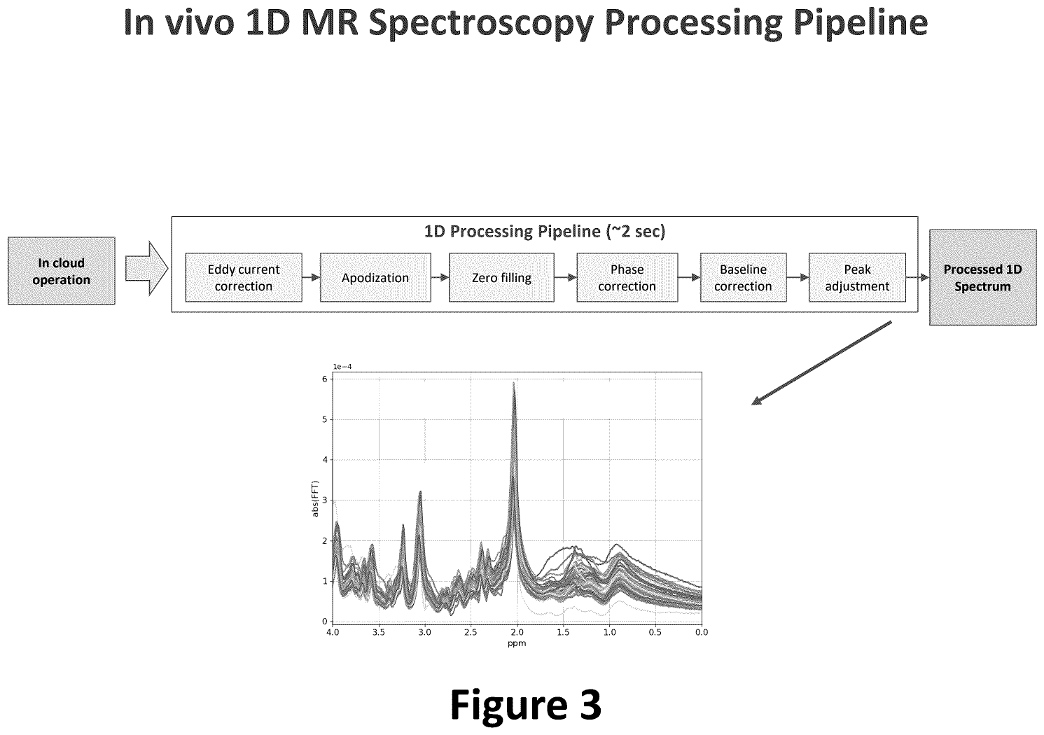

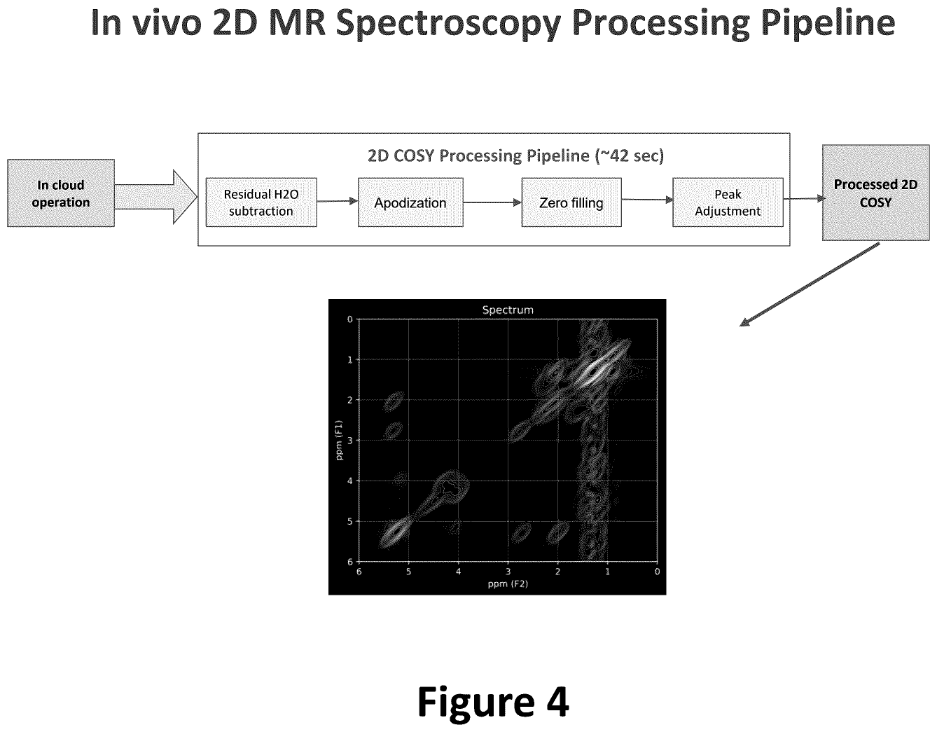

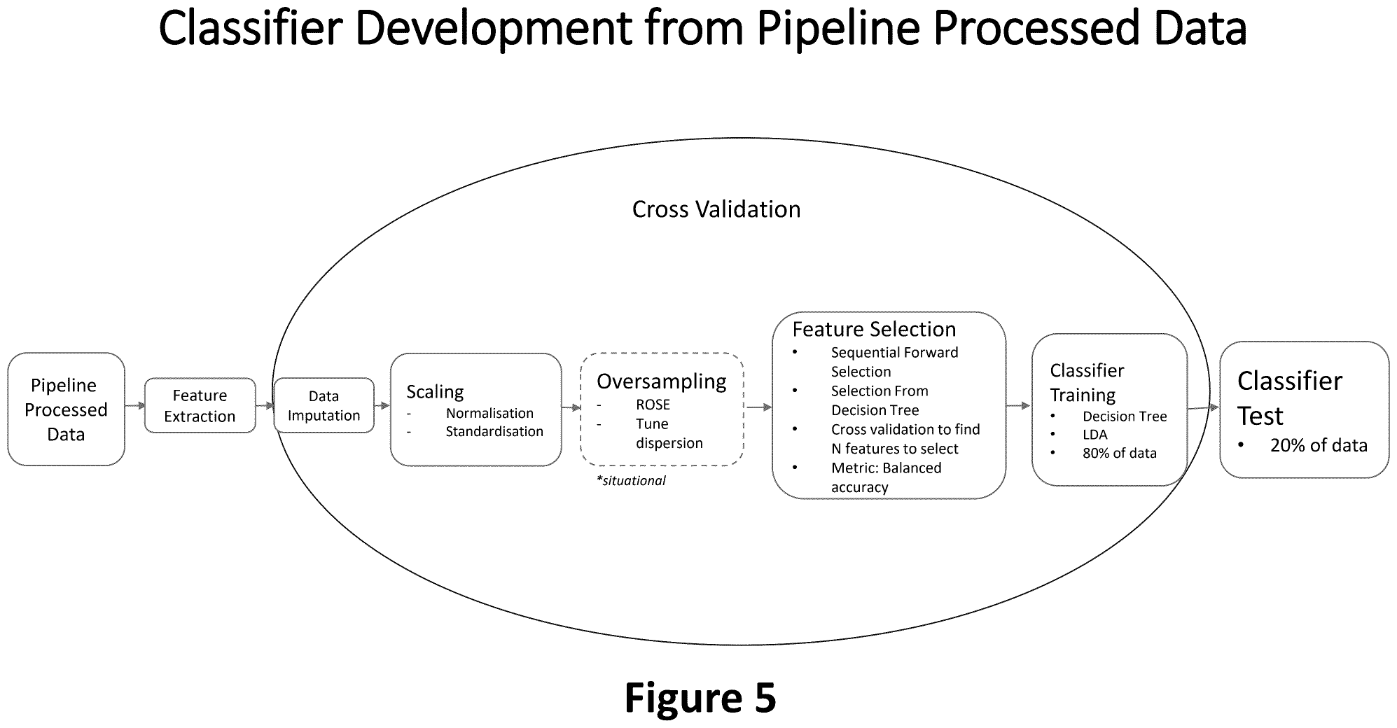

Methods for obtaining a reference database for practicing the invention are shown in FIGS. 3 and 5, which are similar except for the data acquisition initial step, wherein in FIG. 4, data is acquired using Felix 2D data measured by an individual, and in FIG. 5, data is acquired using TWIX data post processing data mining. The remaining steps may be the same, and include “leave me out” cross validation method, in which the next step is scaling, including normalization and standardization.

The next step is feature selection, including sequential forward selection, selection from decision tree, cross-validation to find N features to select as preferably maximally discriminating, and metric: balanced accuracy. The next step includes classifier training, including decision tree, linear discrimination analysis (LDA) for 80% of the data. The next step is a classifier test to test 20% of the data other than the 80%.

The results of the special data acquisition and analysis are shown throughout the drawing Figures.

The results show that all patients have apparently healthy, i.e., histologically normal tissue, away from any lesion or tumor, known or unknown. The results show that subjects with lesions, tumor or cancer away from apparently healthy tissue being evaluated will have substantially different results at various peaks relative to those subjects which do not have any lesions, tumors or cancer, and that the results of evaluating the spectral data from only the apparently healthy tissue will effectively reveal the substantial likelihood that the subject under examination does in fact have a lesion, tumor or cancer in an unexamined region which may have been missed by directly examining that region due to small size or obstruction, or a probability that a lesion, tumor or cancer will develop.

Classifiers, with a high level of balanced accuracy (80%+ in cross-validation)
Fatty vs dense breast tissue in cancer-free healthy controls
Apparently Healthy tissue (determined by mammogram, MRI and ultrasound) but in a patient with a tumor in the contralateral breast or in part of the ipsilateral breast well away from the diagnosed cancer. This is named “Switched on Tissue” for:
   Fatty breast tissue
   Dense breast tissue
   A combination
Histologically confirmed malignant tumor

These results have heretofore been unreported and will be a valuable tool by medical practitioners for early detection, and therefore the ability for early treatment for a lesion, tumor or cancer otherwise missed during conventional examination procedures.

The selected biomarkers may be one or more of choline, phosphocholine, glucose, glycine, myo-inositol, glycerol, glutamine, scyllo-inositol, histamine, methylene protons β to COO, methyl-malonic acid (MMA), MMA1, histidine, taurine, creatine, specific parts of lipid or triglyceride, cholesterol, cholesterol ester, creatine, and methine.

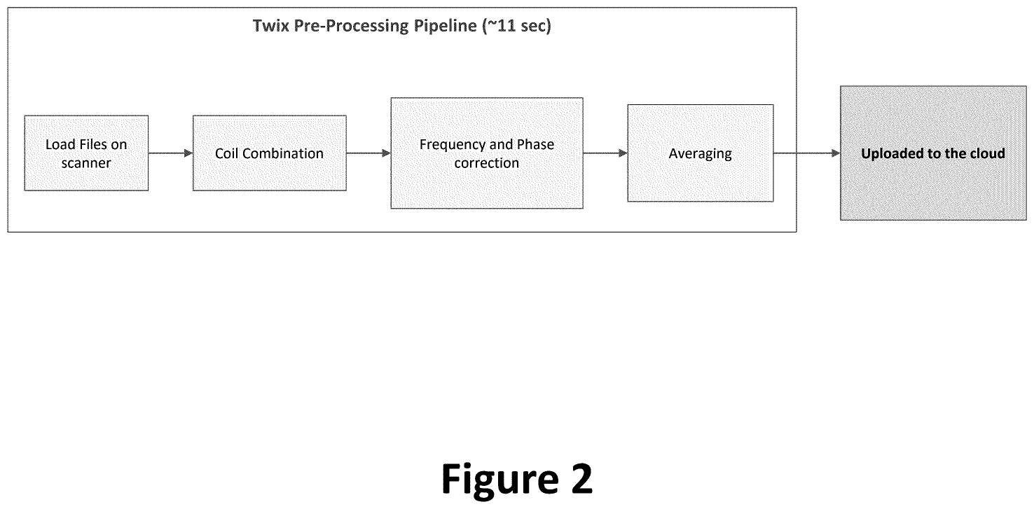

As shown in FIG. 2, the patient data can be automated. New patient data can be obtained in one dimension (1D) and then a second dimension (2D). This is followed by a Fourier Transform (FT) in two dimension and each data point evaluated to determine the best frequencies to distinguish between and produce classifications for:
1. Breast density, i.e., fatty or dense;
2. Level of tumor promotor MMA; and
3. Risk for a cancer developing by comparison to the “switched on” classifier and level of MMA present

The goal is a classifier for each condition from which a cancer may develop

Classifiers Breast (2D COSY Data)

| | | | Production Classifier | |
|---|---|---|---|---|
| | Baseline Classifier | | TBD | |
| | Cross- | Test | Cross- | Test |
| | Validation | Balanced | Validation | Balanced |
| | Accuracy | Accuracy | Accuracy | Accuracy |
| Healthy fatty vs Healthy | 86.27% | 92.86% | | |
| Dense | | | | |
| Healthy vs Healthy | 82.61% | 96.15% | | |
| tumor bearing | | | | |
| (combined density) | | | | |
| Healthy fatty vs Healthy | 79.10% | 92.85% | | |
| fatty tumor bearing | | | | |
| Healthy dense vs Healthy | 88.30% | 92.86% | | |
| dense tumor bearing | | | | |
| Healthy vs Tumor | 87.38% | 83.65% | | |
| (combined density) | | | | |

Classifier Development Breast from 2D COSY Data

| | Cross-Validation | Test |
|---|---|---|
| | Mean of Balanced | Balanced |
| | Accuracy | Accuracy |
| Healthy non-dense vs Healthy Dense | 84.31% | 92.86% |
| Healthy vs Switched On | 93.22% | 83.65% |
| (combined density) | | |
| Healthy (Non-dense) vs Switched | 92.17% | 50% |
| On (Non-dense) | | |
| Healthy (Dense) vs Switched | 88.30% | 92.86% |
| On (Dense) | | |
| Healthy vs Tumour | 88.26% | 87.82% |
| (Combined Density) | | |
| Healthy (Non-dense) vs | 83.80% | 100% |
| Tumour (Non-dense) | | |
| Healthy (Dense) vs Tumour (Dense) | 88.07% | 100% |

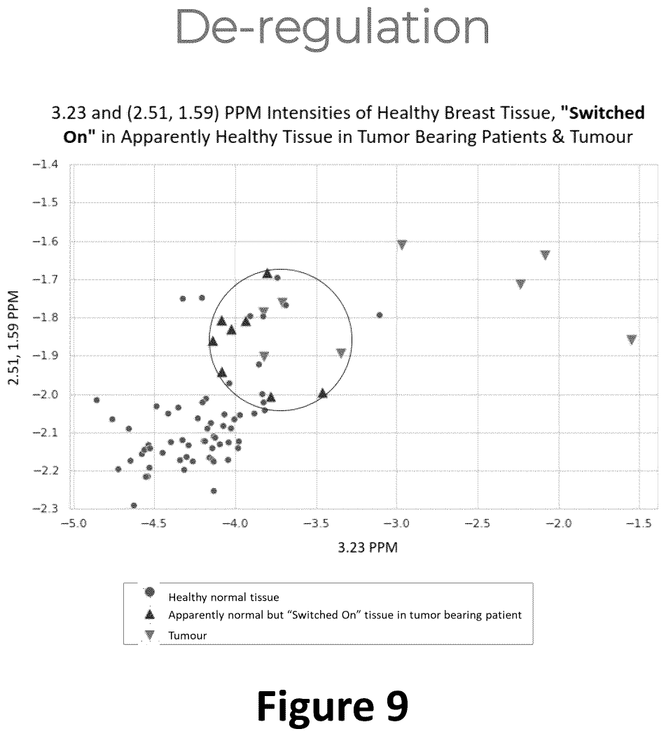

FIG. 9 shows lipid and metabolic de-regulations associated with breast cancer cells forming at an early state, combining lipid and choline changes to map those at risk, the dots indicating chemical changes in apparently healthy tissue in a patient with cancer elsewhere in other (e.g. breast) tissue (called “switched on”) that can be recorded, also increases in choline recorded, the patients have a high risk of developing breast cancer in the near term or already developed cancer not identified by MRI/Mammogram.

Although preferred embodiments have been described, the invention is not limited to these embodiments.

### REFERENCES CITED AND INCORPORATED BY REFERENCE HEREIN

1. D'Orsi C, S. E., Mendelson E, et al., ed. ACR BI-RADS Atlas, breast imaging reporting and data system. 2013, American College of Radiology: Reston, Virginia.
2. Gomes, A. P., et al., Age-induced accumulation of methylmalonic acid promotes tumour progression. Nature, 2020. 585(7824): p. 283-287.
3. Li, Z., et al., Tumor-produced and aging-associated oncometabolite methylmalonic acid promotes cancer-associated fibroblast activation to drive metastatic progression. Nature Communications, 2022. 13(1): p. 6239.
4. Santamaría, G., et al., In vivo assignment of methylmalonic acid in breast tissue using 2D MRS and relationship with breast density, menopausal status and cancer risk. NMR Biomed, 2022: p. e4851 (48-51).
5. Santamaría, G., et al., Breast Tissue Chemistry Measured In Vivo In Healthy Women Correlate with Breast Density and Breast Cancer Risk. Journal of Magnetic Resonance Imaging, 2022. 56(5): p. 1355-1369.

1. A method of enabling a determination of the probability that a subject has or will develop breast cancer, comprising:
using a magnetic resonance spectroscopy device to obtain the level of at least one selected biomarker of a first region of the breast of presumably healthy breast tissue, said level of the selected biomarker having a correlation with the presence or probability of development of a breast tumor in a second region of the breast different from that of the first region of the presumably healthy breast tissue;
comparing and analyzing the level of the selected biomarker from the first region of presumably healthy breast tissue with a reference level of the selected biomarkers which correlates with the probability that the subject has or will likely develop breast tumor in the second region of the breast.

2. The method of claim 1, wherein the spectral data is obtained using one-dimensional (1D) magnetic resonance spectroscopy (MRS).

3. The method of claim 1, wherein the spectral data is obtained using two-dimensional (2D) magnetic resonance spectroscopy (MRS).

4. The method of claim 1, wherein the step of comparing and analyzing comprises measuring the resonances or cross-peaks of the spectral data.

5. The method of claim 1, wherein the step of comparing and analyzing comprises data mining digital points in the spectral data.

6. The method of claim 5, wherein the data mining is obtained from one-dimensional (1D) spectral data.

7. The method of claim 5, wherein the data mining is obtained from two-dimensional (2D) spectral data.

8. The method of claim 1, wherein the selected biomarker(s) is/are selected from the group comprising specific parts of lipid or triglyceride, cholesterol, cholesterol ester, methylene protons β to COO, methine, phosphocholine, glucose, glycine, myo-inositol, glycerol, glutamine, scyllo-inositol, histamine, methyl-malonic acid (MMA), MMA1, histidine, taurine, creatine, creatine and choline.

9. A system for enabling a determination of the probability that a subject has or will develop breast cancer, comprising:
a magnetic resonance spectroscopy device to obtain the level of at least one selected biomarker of a first region of the breast of presumably healthy breast tissue, said level of the selected biomarker having a correlation with the presence or probability of development of a breast tumor in a second region of the breast different from that of the first region of the presumably healthy breast tissue;
a processor for comparing and analyzing the level of the selected biomarker from the first region of presumably healthy breast tissue with a reference level of the selected biomarkers which correlates with the probability that the subject has or will likely develop a breast tumor in the second region of the breast.

10. The system of claim 9, wherein the magnetic resonance spectroscopy device obtains spectral data using one-dimensional (1D) magnetic resonance spectroscopy (MRS).

11. The system of claim 9, wherein the magnetic resonance spectroscopy device obtains spectral data using two-dimensional (2D) magnetic resonance spectroscopy (MRS).

12. The system of claim 9, wherein the processor measures the resonances or cross-peaks of the spectral data.

13. The system of claim 9, wherein the processor mines digital points in the spectral data.

14. The system of claim 13, wherein the processor mines digital points from one-dimensional (1D) spectral data.

15. The system of claim 13, wherein the processor mines digital points from two-dimensional (2D) spectral data.

16. The method of claim 9, wherein the selected biomarker(s) is/are selected from the group comprising specific parts of lipid or triglyceride, cholesterol, cholesterol ester, methylene protons β to COO, methine, phosphocholine, glucose, glycine, myo-inositol, glycerol, glutamine, scyllo-inositol, histamine, methyl-malonic acid (MMA), MMA1, histidine, taurine, creatine, creatine and choline.

17. A method of enabling a determination of the probability that a subject has or will develop cancer in an organ, comprising:
using a magnetic resonance spectroscopy device to obtain the level of at least one selected biomarker of a first region of the organ of presumably healthy tissue, said level of the selected biomarker having a correlation with the presence or probability of development of a tumor in a second region of the organ different from that of the first region of the presumably healthy tissue;
comparing and analyzing the level of the selected biomarker from the first region of presumably healthy tissue with a reference level of the selected biomarkers which correlates with the probability that the subject has or will likely develop a tumor in the second region of the organ.

18. A system for enabling a determination of the probability that a subject has or will develop cancer in an organ, comprising:
a magnetic resonance spectroscopy device to obtain the level of at least one selected biomarker of a first region of the organ of presumably healthy tissue, said level of the selected biomarker having a correlation with the presence or probability of development of a tumor in a second region of the organ different from that of the first region of the presumably healthy tissue;
a processor for comparing and analyzing the level of the selected biomarker from the first region of presumably healthy tissue with a reference level of the selected biomarkers which correlates with the probability that the subject has or will likely develop a tumor in the second region of the organ.
